(19) United States
(12) Patent Application Publication
(10) Pub. No.: US 2024/0012598 A1
(43) Pub. Date: Jan. 11, 2024

(54) COMPUTER SCREEN BACKGROUND MOOD DETECTION COLOR ALTERING METHOD

(71) Applicant: Fenil Patel, Vancouver (CA)

(72) Inventor: Fenil Patel, Vancouver (CA)

(21) Appl. No.: 17/811,155

(22) Filed: Jul. 7, 2022

(51) Int. Cl. G06F 3/14; G06V 40/16; A61B 5/16; A61B 5/00

(52) U.S. Cl. CPC G06F 3/14; G06V 40/174; A61B 5/165; A61B 5/0082; A61B 2576/02

(57) ABSTRACT

A computer screen or application method configured to provide alterations of the color of the screen or application window based upon the detected mood of the computer user. The present invention employs facial mapping utilizing suitable techniques to capture the facial image of the computer user. During use of the software application of the present invention the computer users face is consistently mapped and analyzed in order to detect expression changes. The present invention will determine the potential mood of the computer user based on the facial expression analysis. Ensuing determination of the mood of the computer user, the present invention will change the color of computer screen or an application window displayed thereon. The color change is directed to either maintain a potential mood or alter the mood of the computer user. The present invention executes constant analysis and color changes based on the consistent mapping.

### FIELD OF THE INVENTION

The present invention relates generally to psychological based computer applications, more specifically but not by way of limitation, a computer application that is configured to detect a user's mood through evaluation of facial expressions and provide alteration of a color of a computer screen based on the determined mood.

### BACKGROUND

Artists, interior designers and psychologists have long understood that color can dramatically affect moods, feelings, and emotions. Colors, like features, follow the changes of the emotions. Color is a powerful communication tool and can be used to signal action, influence mood, and even influence physiological reactions. Certain colors have been associated with increased blood pressure, increased metabolism, and eyestrain. Experiments have demonstrated that light could be combined to form other colors. Such as but not limited to, red light mixed with yellow light creates an orange color. Some colors, such as green and magenta, cancel each other out when mixed and result in a white light. The concept of color psychology has become a hot topic in marketing, art, design, and other areas, Researchers and experts have made a few important discoveries and observations about the psychology of color and the effect it has on moods, feelings, and behaviors.

An individual's feelings about color are often deeply personal and rooted in your own experience or culture. For example, while the color white is used in many Western countries to represent purity and innocence, it is seen as a symbol of mourning in many Eastern countries. Color is a very powerful force in our lives. Color affects our bodies and minds and the state thereof. While perceptions of color are somewhat subjective, there are some color effects that have universal meaning. Colors in the red area of the color spectrum are known as warm colors and include red, orange, and yellow. These warm colors evoke emotions ranging from feelings of warmth and comfort to feelings of anger and hostility. Colors on the blue side of the spectrum are known as cool colors and include blue, purple, and green. These colors are often described as calm, but can also call to mind feelings of sadness or indifference. Existing research has found that color can impact people in a variety of surprising ways. Warm-colored placebo pills were reported as more effective than cool-colored placebo pills in one study. Blue-colored streetlights can lead to reduced crime according to anecdotal evidence. Red causes people to react with greater speed and force, something that might prove useful during athletic activities according to researchers. Black uniforms are more likely to receive penalties. Additionally, students were more likely to associate negative qualities with a player wearing a black uniform according to a study that looked at historical data of sports teams and what they were dressed. Studies have also shown that certain colors can have an impact on performance. No one likes to see a graded test covered in red ink, but one study found that seeing the color red before taking an exam actually hurt test performance. While the color red is often described as threatening, arousing or exciting, many previous studies on the impact of the color red have been largely inconclusive. Studies have found that exposing students to the color red prior to an exam has been shown to have a negative impact on test performance.

Accordingly, there is a need for a computer application that is installed on a computer wherein the application is operable to determine a user's mood and change the color of the background of the computer screen based upon the user's mood.

### SUMMARY OF THE INVENTION

It is the object of the present invention to provide a computer application and method that is operable to determine a user's mood and subsequently alter the color of the background of the computer screen wherein the present invention includes utilization of a camera on the computer.

Another object of the present invention is to provide a method that is configured to attempt to alter a computer user's mood through color psychology wherein the present invention utilizes the camera to capture a plurality of data points on the user's face.

A further object of the present invention is to provide a computer application and method that is operable to determine a user's mood and subsequently alter the color of the background of the computer screen wherein the present invention provides an initial determination of a user's mood.

Still another object of the present invention is to provide a method that is configured to attempt to alter a computer user's mood through color psychology wherein the present invention provides a second color for the background of the computer screen ensuing detection of the user's mood.

An additional object of the present invention is to provide a computer application and method that is operable to determine a user's mood and subsequently alter the color of the background of the computer screen wherein the present invention provides detection of a change in the user's facial expression.

Yet a further object of the present invention is to provide a method that is configured to attempt to alter a computer user's mood through color psychology wherein the present invention includes altering the color of the computer screen background subsequent detection of a change in the facial expression of the user.

Another object of the present invention is to provide a computer application and method that is operable to determine a user's mood and subsequently alter the color of the background of the computer screen wherein the present invention can provide color change of the computer screen background or other areas of the computer screen.

An alternative object of the present invention is to provide a method that is configured to attempt to alter a computer user's mood through color psychology wherein the present invention provides analysis of facial micro-expressions of the user of the computer on which the present invention is installed.

Yet a further object of the present invention is to provide a computer application and method that is operable to determine a user's mood and subsequently alter the color of the background of the computer screen wherein the present invention can be embodied as a program or software application and employed on a mobile computing device or a desktop computing device.

To the accomplishment of the above and related objects the present invention may be embodied in the form illustrated in the accompanying drawings. Attention is called to the fact that the drawings are illustrative only. Variations are contemplated as being a part of the present invention, limited only by the scope of the claims.

### BRIEF DESCRIPTION OF THE DRAWINGS

A more complete understanding of the present invention may be had by reference to the following Detailed Description and appended claims when taken in conjunction with the accompanying Drawings wherein:

FIG. 1 is an exemplary computer screen demonstrating a first facial image; and

FIG. 2 is an exemplary computer screen demonstrating a second facial image; and

FIG. 3 is an outline of the method of the present invention.

### DETAILED DESCRIPTION

Referring now to the drawings submitted herewith, wherein various elements depicted therein are not necessarily drawn to scale and wherein through the views and figures like elements are referenced with identical reference numerals, there is illustrated computer screen color change method 100 constructed according to the principles of the present invention.

An embodiment of the present invention is discussed herein with reference to the figures submitted herewith. Those skilled in the art will understand that the detailed description herein with respect to these figures is for explanatory purposes and that it is contemplated within the scope of the present invention that alternative embodiments are plausible. By way of example but not by way of limitation, those having skill in the art in light of the present teachings of the present invention will recognize a plurality of alternate and suitable approaches dependent upon the needs of the particular application to implement the functionality of any given detail described herein, beyond that of the particular implementation choices in the embodiment described herein. Various modifications and embodiments are within the scope of the present invention.

It is to be further understood that the present invention is not limited to the particular methodology, materials, uses and applications described herein, as these may vary. Furthermore, it is also to be understood that the terminology used herein is used for the purpose of describing particular embodiments only, and is not intended to limit the scope of the present invention. It must be noted that as used herein and in the claims, the singular forms “a”, “an” and “the” include the plural reference unless the context clearly dictates otherwise. Thus, for example, a reference to “an element” is a reference to one or more elements and includes equivalents thereof known to those skilled in the art. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

References to “one embodiment”, “an embodiment”, “exemplary embodiments”, and the like may indicate that the embodiment(s) of the invention so described may include a particular feature, structure or characteristic, but not every embodiment necessarily includes the particular feature, structure or characteristic.

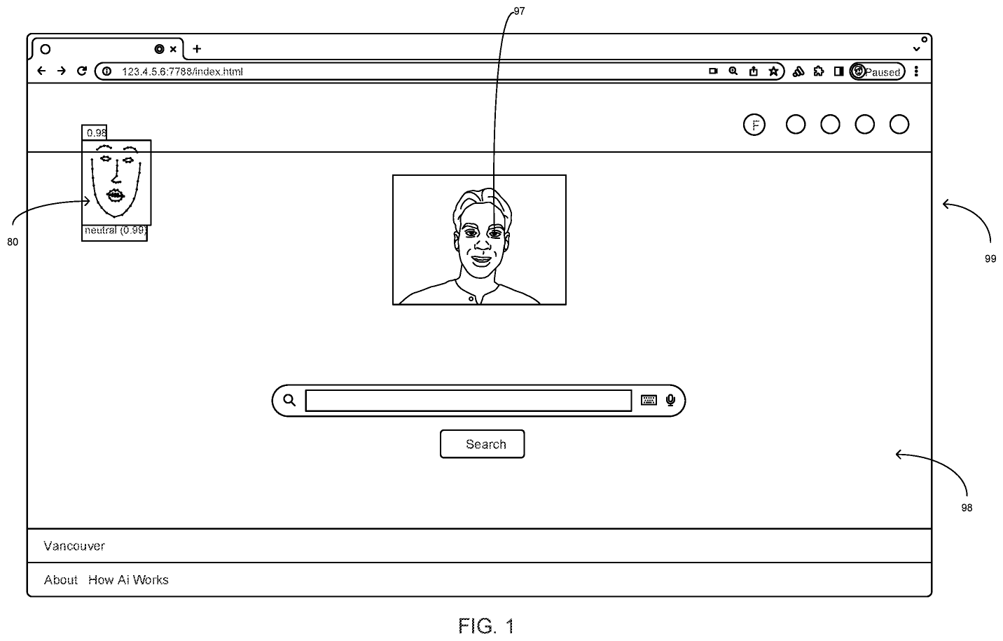

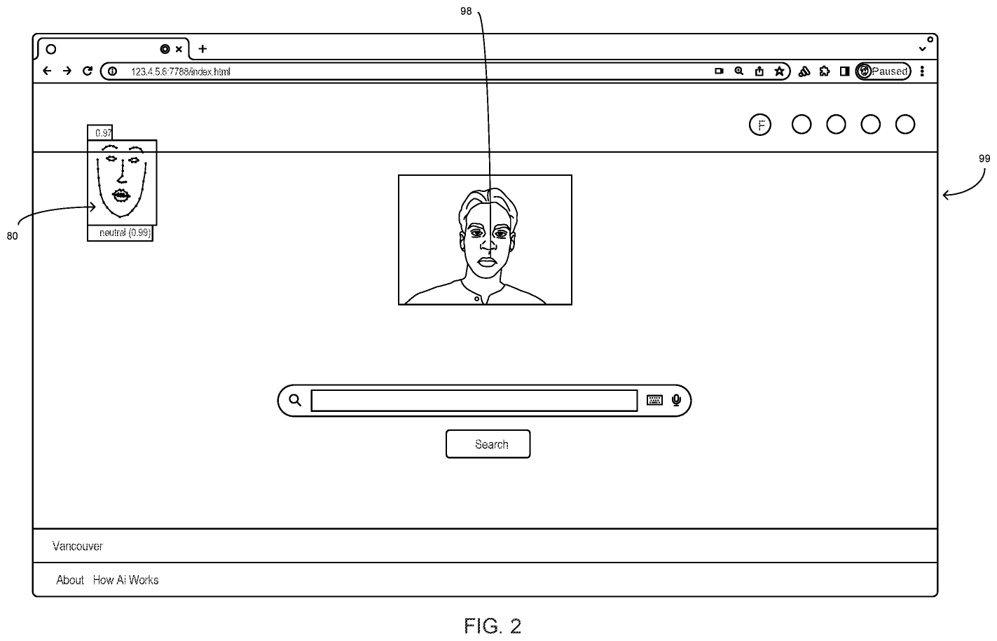

Referring in particular to the Figures submitted herewith, the computer screen color change method 100 is configured to change color of all or a portion of a computer screen 99. It should be understood within the scope of the present invention that the computer screen color change method 100 will change colors inside a particular software application window 98 and/or change the background color of the computer screen 99. Illustrated herein in FIG. 1 and FIG. 2 herein are exemplary facial expressions 97,98 displayed in window 95. It should be understood within the scope of the present invention that window 95 is illustrated herein for the purpose of understanding the present invention and is not displayed to a user during normal use of the computer screen color change method 100. The facial expressions 97, 98 differ and the computer screen color change method 100 employs a facial mapping software component such as but not limited to true depth camera, infrared mapping or face:api.min.js software. It should be understood within the scope of the present invention that various alternate facial mapping and recording could be employed as part of the computer screen color change method 100 in order to obtain a facial map of the user of the computer having the computer screen color change method 100. The computer screen color change method 100 obtains an initial facial expression 97,98 and subsequently monitors for changes thereof as is further discussed herein. The computer screen color change method 100 can include a facial mapping window 80 wherein a user of the computer screen color change method 100 can choose to have an outlined facial diagram displayed in the facial mapping window 80. In the facial mapping window 80, a diagrammed facial outline is displayed along with a plurality of mapping dots thereon. This provides a replica of the current facial expression 97,98 of the user. The diagrammed facial outline will change as the facial expression of the user of the computer screen color change method 100 changes. It should be understood within the scope of the present invention that the facial mapping window 80 is optionally displayed for a user of the present invention and is not displayed by default.

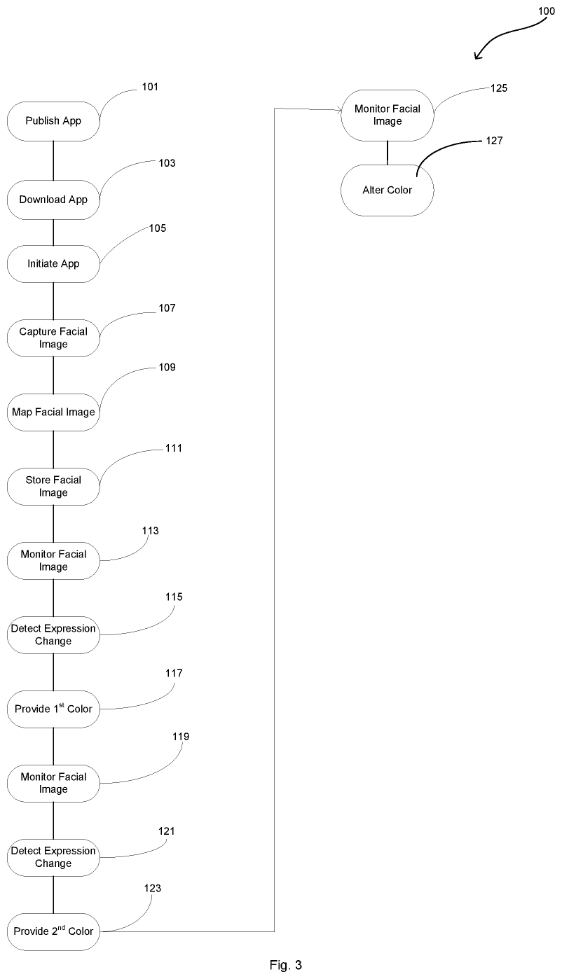

Referring in particular to FIG. 3, an outline of the process facilitated by the computer screen color change method 100 is diagrammed therein. Step 101, the software application of the computer screen color change method 100 is published to at least one website and made available for download. In step 103, a user will download the software application to at least one computing device wherein the computing device will operate the software application of the present invention. It should be understood within the scope of the present invention that the software application of the present invention could be utilized on computing devices such as but not limited to smart phones, tablet computers and desktop computers. Step 105, the user will initiate the software application of the computer screen color change method 100. In step 107, the software application of the present invention will utilize the camera of the computing device to capture an initial facial image. The initial facial image is analyzed and mapped in step 109. Ensuing analyzing and mapping of the initial facial image the software application of the present invention will display a color based on the determined mood of the user derived from the initial facial mapping. The color displayed by the computer screen color change method 100 is a color that either will continue or enhance the potential that the current mood of the user remains if the mood is determined to be pleasant. By way of example but not limitation, if a user's facial mapping determines the user is in a pleasant mood, the computer screen color change method 100 will display warm or cool colors so as to potentially maintain the user's mood. If the initial facial mapping determines the user's mood might be anxious or angry, then appropriate colors are displayed in order to attempt to alter the mood of the user.

In step 111, the initial facial mapping image is stored for use as reference. Step 113, the software application of the present invention provides continuous monitoring and mapping of the facial image of the user of the computing device on which the computer screen color change method 100 is being utilized. In step 115, the software application detects a facial expression change. Step 117, in response to the identified facial expression change the software application of the present invention will provide a first color wherein the first color may either enhance the detected mood as determined by the facial expression change or attempt to alter the mood. In step 119, the software application will continue to monitor the facial image of the user. Step 121, the software application of the computer screen color change method 100 detects a facial image or expression change. In step 123, the software application of the present invention will provide a second color based upon the detected facial image change and potential mood associated therewith wherein the second color will either enhance the potential mood or provide altering thereof. Step 125, the software application provides continuous monitoring of the facial image of the user of the computer screen color change method 100. Step 127, the software application of the computer screen color change method 100 will periodically alter the color of the computer screen and/or software application based upon detected changes in the facial expression of the user.

It is contemplated within the scope of the present invention that the computer screen color change method 100 could be provided in alternate embodiments wherein the alternate embodiments are also determining moods and either changing or continuing based on a color output. By way of example but not limitation, a contemplated alternate embodiment would include a standalone camera wherein the camera is operably coupled to at least one light. The standalone camera would provide capturing of the facial maps and the software as discussed herein would be employed to provide an analysis and determination of the mood of the individual proximate the standalone camera. The camera would transmit a signal to the at least one light so as to provide ambient color in places such as but not limited to a room or a vehicle in order to achieve the desired effect as discussed herein.

In the preceding detailed description, reference has been made to the accompanying drawings that form a part hereof, and in which are shown by way of illustration specific embodiments in which the invention may be practiced. These embodiments, and certain variants thereof, have been described in sufficient detail to enable those skilled in the art to practice the invention. It is to be understood that other suitable embodiments may be utilized and that logical changes may be made without departing from the spirit or scope of the invention. The description may omit certain information known to those skilled in the art. The preceding detailed description is, therefore, not intended to be limited to the specific forms set forth herein, but on the contrary, it is intended to cover such alternatives, modifications, and equivalents, as can be reasonably included within the spirit and scope of the appended claims.

What is claimed is:

1. A method configured to provide changing of colors displayed on a computer screen wherein the method comprises the steps of:
providing a software application, said software application configured to be downloaded to a computing device;
capturing an initial facial image map, wherein the software application utilizes a camera on the computing device to capture the initial facial image map of a user of the computing device;
analyzing the initial facial image map, wherein the software application analyzes the initial facial image map;
determining a mood of the user, wherein the software application of the present invention determines the mood of the user based on the initial facial image map;
displaying a color, wherein the software application will display a color on the computing device based on the mood of the user.

2. The method configured to provide changing of colors displayed on a computer screen as recited in claim 1, and further including the step of capturing a subsequent facial image map.

3. The method configured to provide changing of colors displayed on a computer screen as recited in claim 2, and further including the step of analyzing the subsequent facial image map.

4. The method configured to provide changing of colors displayed on a computer screen as recited in claim 3, and further including the step of determining a mood based on the subsequent facial image map.

5. The method configured to provide changing of colors displayed on a computer screen as recited in claim 4, and further including the step of altering the color displayed on the computing device, wherein the color is altered based on the mood determined.

6. The method configured to provide changing of colors displayed on a computer screen as recited in claim 5, wherein the altered color is operable to maintain the mood determined.

7. The method configured to provide changing of colors displayed on a computer screen as recited in claim 5, wherein the altered color is operable to change the mood determined.

8. The method configured to provide changing of colors displayed on a computer screen as recited in claim 5, and further including the step of monitoring the facial image of the user so as to analyze for changes in facial expression.

9. A method operable to either influence or maintain a mood of a user of a computing device wherein the method comprises the steps of:
providing a software application, said software application configured to be downloaded to a computing device;
displaying a first color on the computing device, wherein the software application displays a first color on the computing device;
capturing an initial facial image map, wherein the software application utilizes a camera on the computing device to capture the initial facial image map of a user of the computing device;
analyzing the initial facial image map, wherein the software application analyzes the initial facial image;
determining a mood of the user, wherein the software application of the present invention determines the mood of the user based on the initial facial image map;
displaying a color, wherein the software application will display a second color on the computing device based on the mood of the user, wherein the second color is different than the first color;
capturing an additional facial image map, wherein the camera is utilized to capture an additional facial image map;
analyzing the additional facial image map, wherein the software application is utilized to analyze the additional facial map and determine the mood of the user based on the facial expression of the user; and
changing the color displayed on the computing device, wherein the software application changes the color displayed on the computing device based on the facial expression of the user.

10. The method operable to either influence or maintain the mood of the user of a computing device as recited in claim 9, and further including the step of continuing to capture and analyze facial image maps of the user, wherein the software application provides capture and analysis of the facial image maps during use of the computing device by the user.

11. The method operable to either influence or maintain the mood of the user of a computing device as recited in claim 10, and further including the step of changing the color displayed on the computing device, wherein the color is changed based upon detection in changes of the facial image map of the user.

12. The method operable to either influence or maintain the mood of the user of a computing device as recited in claim 11, wherein the color displayed on the computing device is operable to change the mood of the user.
